(19) United States
(12) Patent Application Publication
(10) Pub. No.: US 2008/0292688 A1
(43) Pub. Date: Nov. 27, 2008

(54) Liposome and preparation method of the same

(75) Inventors: Ae-June Wang, Hsinchu City (TW); Pei-Lin Wang, Chiai City (TW); Wan-Ko Chen, Hsinchu City (TW); Chi-Heng Jian, Jiaosi Township (TW)

Correspondence Address: BACON & THOMAS, PLLC, 625 SLATERS LANE, FOURTH FLOOR, ALEXANDRIA, VA, 22314-1176, US

(73) Assignee: Industrial Technology Research Institute, Judung Township (TW)

(21) Appl. No.: 12/076,295

(22) Filed: Mar. 17, 2008

Related U.S. Application Data
(63) Continuation of application No. 11/024,799, filed on Dec. 30, 2004, now abandoned.

(30) Foreign Application Priority Data
Dec. 31, 2003 (TW) 092137778
Dec. 29, 2004 (TW) 093141114

(51) Int. Cl. A61K 9/127; A61K 31/355; A61K 31/07; A61K 31/05; A61K 31/24

(52) U.S. Cl. 424/450; 514/458; 514/725; 514/731; 514/539

(57) ABSTRACT

The present invention relates to a composition and a method for preparing a liposome, the liposome including a lipid bilayer and an aqueous core contains a hydrophobic or a hydrophilic drug and a component—Vitamin E derivative (d-α tocopheryl polyethylene glycol 1000 succinate; TPGS). TPGS is able to increase the encapsulation efficiency of drug in liposome as well as to enhance the stability of drug in liposomes. Such liposome is capable to increase the skin permeation of drugs. The preparation method comprises the following steps: (1) adding the drug to a Vitamin E derivative solution to form a mixture; and (2) adding at least one phosphatidyl choline to the mixture, after hydration from either sonication or homogenization.

This application is a continuation application of parent U.S. application Ser. No. 11/024,799, filed Dec. 30, 2004, (of which the entire disclosure of the parent application is hereby incorporated by reference).

### BACKGROUND OF THE INVENTION

1. Field of the Invention

The present invention relates to a composition and a preparation method of a liposome that enhances drug encapsulation efficiency, drug stability, and skin permeability. A preparation method of liposomes that encapsulate hydrophobic drugs, which is selected from the group comprises of all-trans retinoic acid (RA), 4-phenylbutyric acid (PBA), and diclofenac diethylamine.

2. Description of the Related Prior Art

All-Trans Retinoic Acid (abbreviated as RA) is one of the most effective drugs for acne treatment currently. Its functional mechanism is that the synthesis of an active substance after RA clearance in the system and this active substance will induce the epithelial cell proliferation. Due to the proliferation of epithelial cells, keratosis becomes abnormal whereby keratinocytes tend to become loose and less intact, resulting in acne tissue falling off the treated area and thus excellent acne treatment can be achieved. However, some side effects in the course of RA therapeutic session, skin has tendency to peel off, inflame, swell, and also has other unpleasant consequence. The application of using liposomes as a carrier to encapsulate RA may prevent RA in direct interaction with skin, thereby reduce skin irritation. RA liposomes also extend the residence time of drug in the dermal layer and reduce the systemic effect to enhance the drug efficacy in the skin. RA is poorly soluble in water, therefore a non-irritating solubilizer is necessary to apply to increase both RA solubility and the drug concentration in liposomes. Increasing potential risk of fetal deformity in pregnancy while RA is in systemic circulation. The use of RA liposome tends to reduce the amount of RA in systemic circulation by retaining most RA under both the dermis and epidermis of the skin.

Both of 4-phenylbutyric acid, a skin cancer and wound healing drug for treating skin cancer, and diclofenac, a non steroid anti-inflammatory and allergestic drug, may cause potential side effects on skin such as allergic or non-allergic dermatitis, pimples, skin blush, dropsy, scaling, and other symptoms such as the stomach irritation. Therefore, it is desirable to use a liposomes carrier to diminish skin irritation and allow the drugs to remain in the skin (local area) longer for better therapeutic effect and to reduce systemic side effect in the body.

Liposomes are one of the most potential drug carriers available currently. The composition of liposomes contains both hydrophobic bilayer, which may encapsulate hydrophobic substance, and an aqueous core, which may encapsulate hydrophilic substance. The uniqueness for the present invention is that with the use of liposome to encapsulate both hydrophobic and hydrophilic substances. Therapeutic effect is enhanced due to the skin permeability is also enhanced. To prolong RA liposome retention in the dermis and epidermis in the skin will minimize the side effect from systemic circulation. To achieve this goal, the encapsulation efficiency and stability of drug address a major role in present invention.

In according to the past reviews, an amphiphilic Vitamin E derivative (d-α tocopheryl polyethylene glycol 1000 succinate abbreviated here as TPGS) is used as the solubilizer for Paclitaxel in the oral delivery, or as an ingredient in cosmetic preparation, skin medication, and blood clotting. However, the most TPGS applications are irrelevant to the present invention. In the present invention, TPGS hereinafter is solely functioned for active ingredient of topical-used liposome, drug absorption enhancement, or functioned as a solubilizer for insoluble drugs. Due to amphiphilic characteristic of TPGS, that is one hydrophobic portion, Vitamin E, and other hydrophilic portion, polyethylene glycol 1000, liposomes that consist of TPGS which is utilized as a surfactant between the liposome bilayer and core, and will enhance the stability for both hydrophilic or hydrophobic drugs. Furthermore, it will increase the amount of drug encapsulated in the liposome for clinical application.

### SUMMARY OF THE INVENTION

The main objective of the present invention is to provide a composition of a liposome and a method for preparing liposomes. It is able to encapsulate either hydrophobic or hydrophilic drugs within liposomes and increase solubility of hydrophobic drugs. The long-term stability for encapsulating either hydrophobic or hydrophilic drugs in the liposome is also enhanced. Another objective of the present invention is to provide a novel type of liposomes, which are encapsulating either hydrophobic or hydrophilic drug, that skin permeation, encapsulation efficiency, and long term stability are enhanced. Liposomes, that comprises with TPGS, encapsulate either the hydrophobic or hydrophilic drug, that the localized administration of the drug and reduction of skin irritation can be achieved.

To accomplish prescribe objectives, the present invention describes a liposome composition and a preparation method, which includes phospholipids bilayer and an aqueous core; the liposome comprise either a hydrophobic or hydrophilic drug and TPGS (d-α tocopheryl polyethylene glycol 1000 succinate), and the protocols for liposomes preparation is in the following:

First the prescribed TPGS is dissolved in a solvent, such as water, ethanol, methanol, or 2-propanol to obtain a TPGS solution. In addition, a hydrophobic drug (All-Trans Retinoic Acid) is added to the TPGS solution and stirred until dissolved. Phosphatidyl choline as well as other ingredients, such as cholesterol and Vitamin E, (an antioxidant which may prevent accidental oxidation of the liposome or drug), is added in a pre-determined amount and followed by hydration and sonication to produce the liposomes that is comprising of TPGS.

TPGS is obtained by esterification of d-α-tocopheryl acid succinate with polyethyleneglycol 1000. The HLB value of TPGS (hydrophile-lipophile balance) lies between 15-19, indicating that TPGS is a surfactant, which is well soluble and capable of emulsifying hydrophobic drugs. Therefore, TPGS is added with ethanol to increase the solubility of the hydrophobic drug in according to the present invention. For example, All-Trans Retinoic Acid (abbreviated as RA), the results that are indicate in Table 1, the solubility of RA in ethanol increases 10 times when 20% (w/w) TPGS is added.

TABLE 1
Solubility of RA in Various Solvents

| Solvents (Volume %) | mg/mL | % |
|---|---|---|
| PBS | <0.0001 | — |
| 2.25% Glycerin | 0.004 | 0.0004 |
| Ethanol | 1 | 0.1 |
| 20% TPGS in Ethanol | 10 | 1 |
| 40% TPGS in Ethanol | 16.7 | 1.67 |
| Polyethylene Glycol | 9.7 | 0.97 |
| PEG400(Molecular Weight 400) | | |
| 50% Polyethylene Glycol (Molecular | 0.044 | 0.0044 |
| Weight 400) PEG400 + 50% PBS | | |
| 40% Polyethylene Glycol (Molecular | 0.053 | 0.0053 |
| Weight 400) PEG400 + 60% PBS | | |
| 30% Polyethylene Glycol (Molecular | 0.005 | 0.0005 |
| Weight 400) PEG400 + 70% PBS | | |
| 20% Polyethylene Glycol (Molecular | <0.0001 | — |
| Weight 400) PEG400 + 80% PBS | | |
| 10% Polyethylene Glycol (Molecular | <0.0001 | — |
| Weight 400) PEG400 + 90% PBS | | |

PBS: phosphate buffer saline (prepared by inventor) is composed of (Na₂HPO₄ + NaH₂PO₄ + NaCl) in deionized water, concentration: 10 mM

The purpose of this invention is to increase the TPGS concentration from 20% to 40%, thus it increase RA solubility in ethanol from 1 mg/mL to 16.7 mg/mL. Without TPGS addition, the solubility of RA is less than 1 mg/mL.

### BRIEF DESCRIPTION OF THE DRAWINGS

FIG. 1 of the present invention shows RA liposome encapsulation efficiency versus storage time graph of examples 5 and 9.

FIG. 2 of the present invention shows RA liposome encapsulation efficiency versus storage time graph of examples 10 to 13.

FIG. 3 of the present invention shows RA liposome encapsulation efficiency versus storage time graph of examples 14 to 15.

FIG. 4 of the present invention shows PBA liposome encapsulation efficiency versus storage time graph of examples 16 to 18.

### DETAILED DESCRIPTION OF THE PREFERRED EMBODIMENTS

There are eighteen examples to demonstrate the technical breakthrough for this invention.

### Example 1

### Soybean Phosphatidyl Choline (Abbreviated as SPC) Liposome Formulation

Formulation of the present example is illustrated in the following:

TABLE 2
Various Ingredients Composition of Example 1

| | SPC | Cholesterol | Vitamin E | TPGS | RA |
|---|---|---|---|---|---|
| Weight percent | 10 | 1.12 | 0.5 | 2 | 0.1 |
| Weight [gram] | 1 | 0.112 | 0.05 | 0.2 | 0.01 |

| | |
|---|---|
| [solvent/water] | 10/90 |
| Volume Ratio | |

First, 1 g of SPC, 1.12 g of Cholesterol and 0.05 g of Vitamin E are dissolved in ethanol and the solution is stirred until dissolved completely.

In addition, 0.2 g of TPGS and 0.01 g of RA are also mixed and stirred until dissolved completely in ethanol.

8.4 mL of 2.25% glycerin is pipetted into a hydration cell, while the internal temperature is controlled at 25° C. by water circulation, then the resultant solution of SPC, cholesterol, Vitamin E, TPGS, and RA is injected and hydration for an hour.

Finally sonication is performed with the prepared multi-lamellar vesicles (abbreviate as MLVs) liposome. The solution is slightly transparent yellow, which is the target RA liposome.

TABLE 3
Compositions and Properties of Examples 2 to 12

| | | | | | | | | | Particle | [RA] % | [RA] % |
|---|---|---|---|---|---|---|---|---|---|---|---|
| Weight | | | | | | | | | size | (Day | (Day |
| [%] | E 80 | HSPC | Lipoid SPC-3 | DMPG | Cholesterol | Vitamin E | RA | Solvent | (nm) | 0) | 7) |
| Comparative | 0.1 | | | | | 0.0003 | 0.05 | Dichloromethane | Failed | Failed | * |
| Example 1 | | | | | | | | | | | |
| Comparative | | | 10 | | 10 | 0.03 | 0.01 | Chloroform | Failed | Failed | * |
| Example 2 | | | | | | | | | | | |
| Comparative | | 10 | | | 10 | 0.03 | 0.01 | Chloroform | Failed | Failed | * |
| Example 3 | | | | | | | | | | | |
| Comparative | 1 | | | 0.48 | | | 0.02 | Chloroform | 93.3 | 0.020% | Failed |
| Example 4 | | | | | | | | | | | |
| Comparative | 5 | | | | 0.56 | | 0.064 | Chloroform | 92 | 0.053% | Failed |
| Example 5 | | | | | | | | | | | |

The results of table 3 indicate that the formulation, without addition of TPGS, will result in either failure of liposomes formation or decomposed within seven days.

TABLE 4
Compositions and Properties of Comparative Example 6 to 10 and
Example 2 to 3

| | | | | | | | Particle | | |
|---|---|---|---|---|---|---|---|---|---|
| Weight | | | | | | | size | [RA]% | [RA]% |
| [%] | E 80 | Cholesterol | Vitamin E | TPGS | RA | Solvent | (nm) | (Day 0) | (Day 7) |
| Comparative | 1 | 0.1 | | | 0.01 | 10% E | Failed | Failed | * |
| Example 6 | | | | | | | | | |
| Comparative | 1 | 0.56 | | | 0.1 | 10% E | Failed | Failed | * |
| Example 7 | | | | | | | | | |
| Example 2 | 1 | 0.56 | | 2 | 0.1 | 10% E | 73.4 | 0.085% | 0.046% |
| Comparative | 5 | 0.25 | 0.25 | | 0.15 | 10% E | 82.3 | 0.130% | Failed |
| Example 8 | | | | | | | | | |
| Comparative | 5 | 0.25 | 0.25 | | 0.13 | 10% E | 87.9 | 0.980% | Failed |
| Example 9 | | | | | | | | | |
| Comparative | 5 | 0.25 | 0.25 | | 0.18 | 10% E | Failed | Failed | * |
| Example | | | | | | | | | |
| 10 | | | | | | | | | |
| Comparative | 5 | 0.28 | | | 0.064 | 10% E | Failed | Failed | * |
| Example | | | | | | | | | |
| 11 | | | | | | | | | |
| Example 3 | 5 | 0.28 | | 2 | 0.1 | 10% E | 79.7 | 0.083% | 0.083% |

The result of Table 4 shows, in comparison between comparative examples, 6 and 7, 8 and 11, that is, without added TPGS in the formulation, the failure of liposomes formation and decomposed in seven days.

For example 2, the liposomes preparation can be achieved due to the addition of TPGS in the formulation. The drug concentration is increased to 0.085%.

For example 3, the stability of liposomes has enhanced due to the addition of TPGS in the formulation. The RA concentration remains the same after seven days.

### For Example 4 to 9

In according to the method of example 1 preparation. It follows the same method of formulation preparation in Table 3.

TABLE 5
Compositions and Properties of Examples 4 to 9

| Weight | | | | | | | Particle | |
|---|---|---|---|---|---|---|---|---|
| [%] | SPC | Cholesterol | Vitamin E | TPGS | RA | Solvent | Size (nm) | [RA]% |
| Example 4 | 5 | 0.28 | — | 2 | 0.1 | 10% E | 82.6 | 0.096% |
| Example 5 | 10 | 1.12 | 0.5 | 2 | 0.1 | 10% E | 53.3 | 0.102% |
| Example 6 | 10 | 1.12 | 0.5 | 2 | 0.1 | 10% IPA | 66.8 | 0.073% |
| Example 7 | 10 | 1.12 | 0.5 | 2 | 0.2 | 10% IPA | 31.0 | 0.143% |
| Example 8 | 10 | 0.56 | 0.5 | 2 | 0.15 | 10% E | 64.5 | 0.124% |
| Example 9 | 5 | 0.56 | — | 2 | 0.1 | 10% E | 92 | 0.083% |

10% E: 10% ethanol(v/v) in total solution.
10% IPA: 10% 2-propanol(v/v) in total solution.

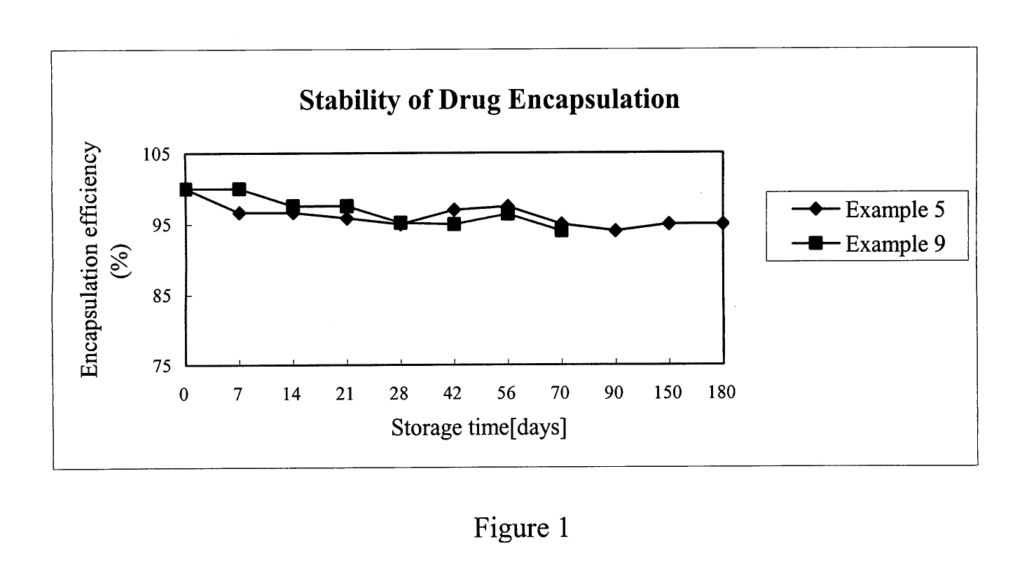

FIG. 1 illustrates the encapsulation efficiency of RA versus storage time of examples 5 and 7. As shown in FIG. 1, the present invention employing TPGS as composition of RA liposome prolong the stability of RA within the liposome to 180 days while greatly enhancing the encapsulation efficiency of RA.

### Examples 10 to 13

### Formulation of Egg Phosphatidyl Choline Liposome

The method of liposome preparation for examples 10 to 13 is using the same method as in example 1. The only difference is that SPC is being replaced by E60 (EPC of 60% purity). The composition and properties for previous 4 formulations are listed in Table 6.

TABLE 6
Compositions and Properties of Examples 10 to 13

| Weight | | | | | | | Particle | |
|---|---|---|---|---|---|---|---|---|
| [%] | E60 | Cholesterol | Vitamin E | TPGS | RA | Solvent | Size (nm) | [RA]% |
| Example 10 | 10 | 0.56 | — | 4 | 0.16 | 10% E | 103.4 | 0.130% |
| Example 11 | 10 | 0.56 | — | 2 | 0.2 | 20% E | 50.6 | 0.172% |
| Example 12 | 10 | 1.12 | 0.5 | 2 | 0.15 | 10% E | 115.9 | 0.105% |
| Example 13 | 10 | 1.12 | — | 2 | 0.1 | 10% E | 111.6 | 0.075% |

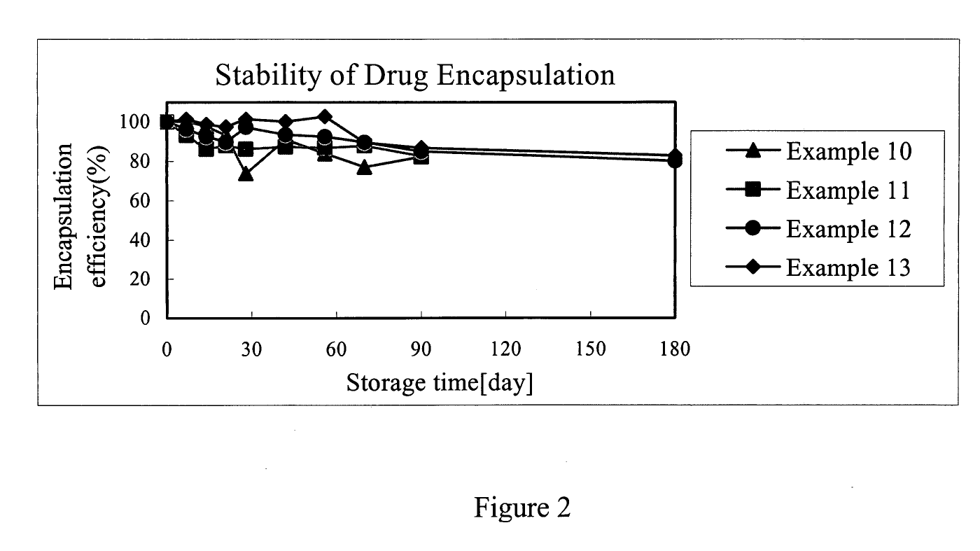

The results of the above examples are shown in FIG. 2, which illustrates the encapsulation efficiency versus storage time of examples 10 to 13. As shown in the FIG. 2, the present invention employing TPGS as composition of RA liposome prolong the stability of RA within the liposome to 180 days while greatly enhancing the encapsulation efficiency of RA.

### Examples 14 to 15

The method of liposome preparation of examples 14 to 15 is the identical method as in example 1, only soybean PC is being replaced by hydrogenated soy phosphatidyl choline (abbreviated as HSPC) and soybean PC together, or employing both SPC and HSPC without the use of Vitamin E. Formulation compositions and liposome properties of the above mentioned examples are shown in Table 7.

TABLE 7
Compositions and Properties of Examples 14 to 15

| | | | | | | | Particle | |
|---|---|---|---|---|---|---|---|---|
| Weight | | HSPC- | | | | | Size | |
| [%] | SPC | 75 | Cholesterol | TPGS | RA | Solvent | (nm) | [RA]% |
| Example 14 | 5 | 2 | 0.56 | 2 | 0.1 | 10% E | 70.1 | 0.092% |
| Example 15 | 5 | 3.5 | 0.56 | 2 | 0.1 | 10% E | 87.8 | 0.087% |

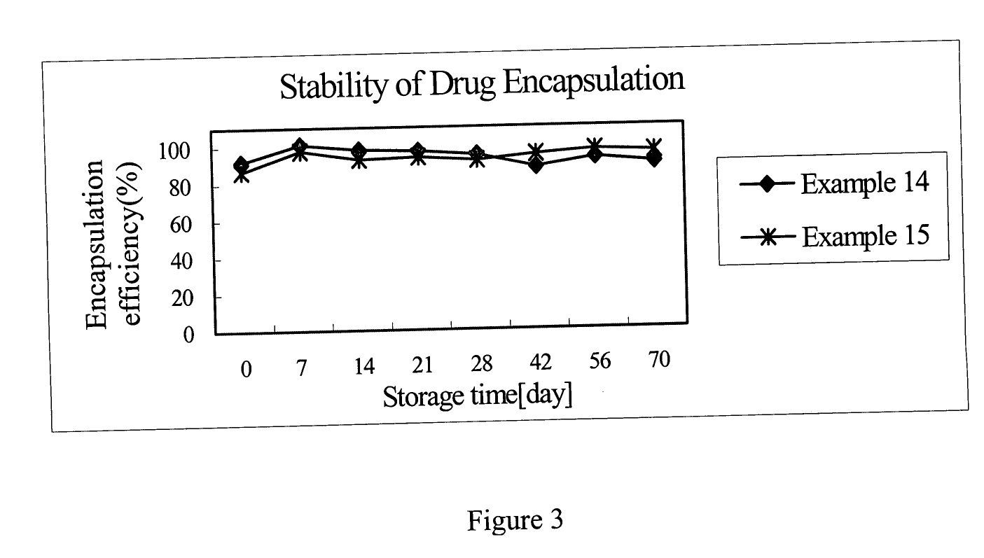

FIG. 3 illustrates the encapsulation efficiency versus storage time graph of examples 14 to 15. As shown in the FIG. 3, the present invention employing TPGS as composition of RA liposome prolong the stability of RA within the liposome to 70 days while greatly enhancing the encapsulation efficiency of RA.

### Examples 16 to 21

Formulation of soybean PC liposome encapsulated with 4-phenylbutyric acid (abbreviated as PBA). As in the preparation method of example 1, and in accordance with liposome formulation preparation of Table 8, compositions and properties of all examples are illustrated in Table 8.

TABLE 8
Compositions and Properties of Examples 16 to 21

| | | | | | | | Particle | |
|---|---|---|---|---|---|---|---|---|
| Weight | | | | | PBA | | Size | |
| [%] | SPC | Cholesterol | Vitamin E | TPGS | (%) | Solvent | (nm) | [PBA] % |
| Example 16 | 10 | 1.12 | 0.5 | 0 | 1.0 | 10% E | Failed | Failed |
| Example 17 | 10 | 1.12 | 0.5 | 1 | 1.5 | 10% E | Failed | Failed |
| Example 18 | 10 | 1.12 | 0.5 | 2 | 0.5 | 10% E | 64.4 | 0.45% |
| Example 19 | 10 | 1.12 | 0.5 | 2 | 1.0 | 10% E | 71.9 | 0.97% |
| Example 20 | 10 | 1.12 | 0.5 | 2 | 1.5 | 10% E | 73.3 | 1.5% |
| Example 21 | 10 | 1.12 | 0.5 | 4 | 1.5 | 10% E | 74.3 | 1.5% |

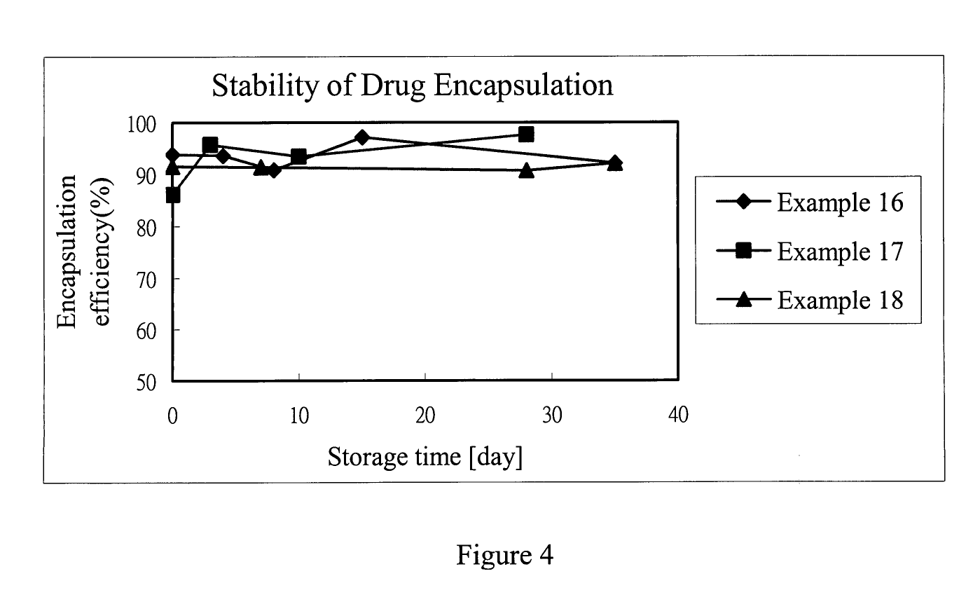

PBA encapsulated efficiency versus storage time of examples 16 to 21 is illustrated in FIG. 4. From the graph interpretation, the present invention employs TPGS as the composition of PBA liposome, which enhances encapsulation efficiency while promote the stability of PBA liposome.

### Example 22

Formulation of soybean PC liposome encapsulated with Diclofenac diethylamine.

Formulation of the present example is shown in Table 9:

TABLE 9
Various Compositions of Example 22

| | | | | Diclofenac |
|---|---|---|---|---|
| | SPC | Cholesterol | TPGS | diethylamine |
| Mole Ratio | 10 | 1 | 1 | 0.1 |
| Weight[gram] | 0.42 | 0.02 | 0.0653 | 0.01 |

| | |
|---|---|
| [Solute/Water] | 1/1 |
| Volume Ratio | |

First, 0.42 g of SPC, 0.02 g of cholesterol, and 0.0653 g of TPGS are dissolved in 0.5 mL of 1% Diclofenac diethylamine solution. Following by a grinding technique, the solution is to dissolve completely to a paste-like mixture.

Hydration is processed by adding PBS solution with paste-like mixture for an hour at room temperature with the volumetric ratio of 1:1 for solute to water. When the hydration is terminated, the yield product is a milky yellow solution, which is Diclofenac diethylamine liposome.

### Examples 23 to 25

As in the manufacture method of example 22, and in accordance with liposome formulation preparation of Table 10, all compositions and properties of examples are listed below in Table 10.

TABLE 10
Compositions and Properties of Examples 23 to 25

| | | | | drug/PC (molar | Particle Size | [Diclofenac |
|---|---|---|---|---|---|---|
| Molar ratio | SPC | Cholesterol | TPGS | ratio) | (nm) | diethylamine] % |
| Example 23 | 10 | 1 | 0.06 | 1/9 | 279.8 | 0.4 |
| Example 24 | 10 | 1 | 0.12 | 1/5.2 | 277.8 | 0.7 |
| Example 25 | 10 | 1 | 2 | 1/4.7 | 195 | 0.8 |

With the addition of TPGS as described in the above Table 8, solubility of RA can be increased; moreover TPGS can also increase the encapsulation efficiency of liposomes. Other than enhancing the encapsulated stability of RA in the liposome, the above mentioned effects can still be obtained when preparing liposomes with different sources of phosphatidyl choline. Therefore, the technique of adding TPGS in present invented liposomes can be applied widely in formulating liposomes, and it is not limited to phosphatidyl choline of the present example.

It is to be noted that the concentration of TPGS in the TPGS solution is not restricted, however, between 1%-50% (by weight percent) is preferable. Depending on the need, 0.1˜20% (by weight percent) of Vitamin E can also be added to the TPGS solution. The amount of Vitamin E in the TPGS-Vitamin E solution is not restricted, but preferably is 1%-20% of the total solution by weight. Preferable phospholipid that is used in the liposome of the present invention includes, but is not limited to, saturated phosphatidyl choline or unsaturated phosphatidyl choline, for example, hydrogenated natural phospholipid or long chain saturated phospholipid, unsaturated phospholipid or short chain saturated phospholipid. Preferable long chain saturated phospholipid includes, but is not limited to, phosphatidyl choline (PC), phosphatidyl glycerol (PG), phosphatidyl serine (PS), or phosphatidyl ethanolamine (PE). Preferable phosphatidyl choline includes, but is not limited to, Hydrogenated egg phosphatidyl choline (HEPC), and hydrogenated soy phosphatidyl choline (HSPC). Preferable long chain saturated phosphatidyl choline includes, but is not limited to, dipalmitoyl phosphatidyl choline (DPPC), distearyloyl phosphatidyl choline (DSPC), or the combination thereof. Examples of unsaturated phophatidyl choline include, but are not limited to, egg phosphatidyl choline (EPC), soy phosphatidyl choline (SPC), and other synthetic unsaturated PC or natural unsaturated PC. Preferable short chain saturated phosphatidyl choline includes, but not limited to, dilauroyl phosphatidyl choline (DLPC).

### Application Example

### In Vivo Test of Skin Irritation and Hypersensitivity

In vivo test of skin irritation and hypersensitivity of example 5 are obtained in courtesy of US Northview Pacific Laboratories, Inc. The results are shown as the following.

(1) Dermal Sensitization Test

The experimental protocol is performed in according to Northview Standard Operation Procedure 16G-60. Using Buehler method for animal studies, that is, in observation of 10 six-weeks old Albino (guinea pig), each weight is between 300 to 500 g, for seven days. The purpose is to determine any skin allergic reaction in Albino (guinea pig) in contacting with the RA liposome. The result indicates that there is no hypersensitive effect.

(2) Skin Irritation Test

The experimental protocol is performed in according to Northview Standard Operation Procedure 16F-03. Six New Zealand White rabbits, each weight is between 2.5 to 2.8 kg, that each has been treated with 0.5 g/site RA liposomes gel for seven days. Any sign of skin irritation has been observed in 24 and 48 hours time period after the removal of drug patches. The results indicate that there is no skin irritation due to low PIS value. Low PIS values represent low irritation reaction.

TABLE 11
In vivo results of skin irritation test (Primary Irritation Score²)

| | | Commercial | |
|---|---|---|---|
| | | product | |
| Sample name | Example 5 | (RA-cream) | Retin-A gel |
| Erythema and Eschar | 0.7 | 8 | 2.5 |
| Edema | 0 | 0 | 0 |
| Total | 0.7 | 8 | 2.5 |
| Primary Irritation Score² (PIS) | 0.2 | 2 | 1 |

No erythema: 0
Very slight erythema (barely perceptible): 1
Well-defined erythema: 2
Moderate erythema: 3
Severe erythema to eschar formation: 4

### Comparative Example 12

### In Vitro Skin Permeation Test

In order to evaluate the efficacy of present invention, some in vitro skin permeation studies have been performed.

Methods of In Vitro Skin Permeation Study

1. Materials and Reagents:
Skin: cadaver skin
Device of skin permeation: Modified Franz Diffusion Cell

2. Methods:
(1) Preparation of extraction solution: Mix the absolute ethanol with 10 mM, pH 7.4 PBS with the ratio of 1:1 (v/v)
(2) After adding the solution prepared in step (1) into the Modified Franz Diffusion Cell, stick the diffusion cell into the hot plate. Set up the temperature to 32±0.5° C.
(3) Remove of the pretreated and defrost cadaver skin at room temperature. Fix up the skin permeation device on the stainless framework.
(4) Record the time for experiment, and collect the sample at pre-determined time points.
(5) Analyze the collected samples by HPLC, and calculate the flux and the cumulated amount.

According to the methods mentioned above, we proceed to the in vitro skin permeation test of example 3, 10, 11, and 18. The results are showed in table 12.

TABLE 12
In vitro skin permeation results of example 3, 10, 11, 18

| | Permeation efficiency | Permeation efficiency |
|---|---|---|
| | after 8 hours (%) | after 24 hours (%) |
| Example 3 | 49.80 | 54.92 |
| Example 10 | 38.72 | 49.29 |
| Example 11 | 19.79 | 53.33 |
| Example 18 | 38.35 | 76.15 |

As result of table 12, we find the skin permeability differs with various formulations. In these four examples in table 12, the permeability achieve to 50% in 24 hours. We are able to verify our present invention that will enhance the permeation of RA into the skin.

The in vitro skin permeability of example 5 and commercial RA cream are showed in table 13.

TABLE 13
In vitro skin permeation test results of example 5
and commercial RA cream

| | Permeated | | |
|---|---|---|---|
| | amount | Standard | |
| | (ng/cm²) | deviation | repeat |
| Example 5 | 50.01 | 14.93 | N = 4 |
| Tretinoin | 23.14 | 3.62 | N = 4 |
| cream | | | |

According to the results in table 13 that indicate the present invention is able to improve the skin permeation efficiency to 1-fold. Due to the RA encapsulated in liposome could enhance the interaction between the skin and the liposome, liposome is capable of increasing the penetration of RA. As a result of TPGS in our invention, there is a clear evidence of penetration enhancement.

Although the present invention has been explained in relation to its preferred embodiment, it is to be understood that many other possible modifications and variations can be made without departing from the spirit and scope of the invention as hereinafter claimed.

What is claimed is:

1. A method for preparing a liposome, the liposome including a phospholipids bilayer structure and an aqueous core; the liposome comprising a hydrophobic drug and Vitamin E derivative, the method including:
(1) adding the hydrophobic drug to a pre-mixed solution containing a Vitamin E derivative to obtain a mixed solution; and
(2) adding at least one phosphatidyl choline to the mixed solution in step (1) and then sonication or homogenization after hydration to obtain the liposome.

2. The method as claimed in claim 1, wherein the step (2) further includes a step of adding at least one phosphatidyl choline, a cholesterol and Vitamin E to the mixed solution in step (1).

3. The method as claimed in claim 1 or 2, wherein the step (1) further includes a step (1a) of making the Vitamin E derivative solution.
(1a) adding a Vitamin E derivative in solvent to obtain a Vitamin E derivative solution.

4. The method as claimed in claim 3, wherein the Vitamin E derivative solution comprises 1%-50% Vitamin E derivative by weight percent.

5. The method as claimed in claim 3, wherein the method further includes a step (1a) after the step below (1b):
(1b) adding a Vitamin E to the Vitamin E derivative solution to obtain a Vitamin E derivative-Vitamin E solution.

6. The method as claimed in claim 5, wherein the Vitamin E derivative-Vitamin E solution comprises 0.1%-20% Vitamin E by weight percent.

7. The method as claimed 1 to 6, wherein the at least one solution is alcohol.

8. The method as claimed in claimed 7, wherein the at least one solution is methanol, ethanol, or 2-propanol.

9. The method as claimed in claim 8, wherein the at least one solution is ethanol.

10. The method as claimed in claim 1 and 2, wherein the at least one phosphatidyl choline is selected from a group consisting of: hydrogenated natural phospholipid, long chain saturated phospholipid, long chain unsaturated phospholipid, short chain saturated phospholipid, and the combination thereof.

11. The method as claimed in claim 10, wherein the long chain saturated phospholipid is phosphatidyl choline (PC), phosphatidyl glycerol (PG), phosphatidyl serine (PS) or phosphatidyl ethanolamine (PE).

12. The method as claimed in claim 11, wherein the phosphatidyl choline is hydrogenated egg phosphatidyl choline (HEPC) or hydrogenated soy phosphatidyl choline (HSPC).

13. The method as claimed in claim 10, wherein the long chain saturated phosphatidyl choline is dipalmitoyl phosphatidyl choline (DPPC) or distearyloyl phosphatidyl choline (DSPC).

14. The method as claimed in claim 10, wherein the long chain unsaturated phospholipid is egg phosphatidyl choline (EPC), soy phosphatidyl choline (SPC), synthetic unsaturated phosphatidyl choline or natural unsaturated phosphatidyl choline.

15. The method as claimed in claim 10, wherein the short chain saturated phospholipid is dilauroyl phosphatidyl choline (DLPC).

16. The method as claimed in claim 1, wherein the drug comprising a hydrophobic or hydrophilic drug.

17. The method as claimed in claim 16, wherein the hydrophobic drug is selected from the group consisting of: all-trans retinoic acid, 4-phenylbutyric acid and diclofenac diethylamine.

18. A liposome comprising a phospholipid bilayer structure and a aqueous core, the liposome including a hydrophobic drug and a Vitamin E derivative, which is TPGS.

19. The method as claimed in claim 18, wherein the drug comprising a hydrophobic or hydrophilic drug.

20. The liposome as claimed in claim 19, wherein the hydrophobic drug is selected from the group consisting of: all-trans retinoic acid (RA), 4-phenylbutyric acid (PBA), and Diclofenac diethylamine.

21. The liposome as claimed in claim 18, wherein the Vitamin E derivative comprises 1%-50% by weight percent.

22. The liposome as claimed in claim 18, wherein the phospholipids bilayer structure comprises phosphatidyl choline.

23. The liposome as claimed in claim 18, wherein the phospholipids bilayer structure comprises phosphatidyl choline, cholesterol and Vitamin E.

24. The liposome as claimed in claim 18, wherein the at least one phosphatidyl choline is selected from a group consisting of: hydrogenated natural phospholipid, long chain saturated phospholipid, long chain unsaturated phospholipid, short chain saturated phospholipid, and the combination thereof.

25. The liposome as claimed in claim 24, wherein the long chain saturated phospholipid is phosphatidyl choline (PC), phosphatidyl glycerol (PG), phosphatidyl serine (PS) or phosphatidyl ethanolamine (PE).

26. The liposome as claimed in claim 25, wherein the phosphatidyl choline is hydrogenated egg phosphatidyl choline (HEPC) or hydrogenated soy phosphatidyl choline (HSPC).

27. The liposome as claimed in claim 24, wherein the long chain saturated phosphatidyl choline is dipalmitoyl phosphatidyl choline (DPPC) or distearyloyl phosphatidyl choline (DSPC).

28. The liposome as claimed in claim 24, wherein the long chain unsaturated phospholipid is egg phosphatidyl choline (EPC), soy phosphatidyl choline (SPC), synthetic unsaturated phosphatidyl choline or natural unsaturated phosphatidyl choline.

29. The liposome as claimed in claim 24, wherein the short chain saturated phospholipid is dilauroyl phosphatidyl choline (DLPC).
